(19) United States
(12) Patent Application Publication
(10) Pub. No.: US 2018/0342314 A1
(43) Pub. Date: Nov. 29, 2018

(54) SYSTEM AND METHOD FOR MEDICAL IMAGING WORKFLOW MANAGEMENT WITHOUT RADIOLOGY INFORMATION SYSTEMS

(71) Applicant: MARSHFIELD CLINIC HEALTH SYSTEM, INC., Marshfield, WI (US)

(72) Inventors: Jean Tichy, Marshfield, WI (US); Daniel Kayhart, Marshfield, WI (US); Matthew Gokey, Marshfield, WI (US); Sawyer Butterfield, Marshfield, WI (US); Susan Simon-Madej, Marshfield, WI (US); David Wagner, Marshfield, WI (US)

(21) Appl. No.: 15/896,868

(22) Filed: Feb. 14, 2018

Related U.S. Application Data
(60) Provisional application No. 62/459,430, filed on Feb. 15, 2017.

(51) Int. Cl. G16H 30/20; G16H 80/00; G16H 30/40

(52) U.S. Cl. CPC G16H 30/20; G16H 40/20; G16H 30/40; G16H 80/00

(57) ABSTRACT

Systems and methods for managing medical imaging workflow in an in-office or point-of-care setting outside of a conventional radiology workflow are provided. These system and methods allow for efficient medical imaging workflow management without a Radiology Information System (“RIS”). The systems and methods described here can provide a dual workflow functionality, in which images can be acquired and stored following a prepared medical imaging order according to an “orders first” workflow, or images can be acquired and stored without a prepared medical imaging order according to an “images first” workflow. In both instances, the systems and methods described here interact with the Long Term Archive to quarantine images with missing or incorrect metadata, and to update the quarantined images in an efficient manner to avoid orphaned image studies.

### CROSS-REFERENCE TO RELATED APPLICATIONS

This application claims the benefit of U.S. Provisional Patent Application Ser. No. 62/459,430, filed on Feb. 15, 2017, and entitled “SYSTEM AND METHOD FOR MEDICAL IMAGING WORKFLOW MANAGEMENT WITHOUT RADIOLOGY INFORMATION SYSTEMS,” which is herein incorporated by reference in its entirety.

### BACKGROUND

As in-office medical imaging devices (e.g., ultrasound imaging systems) have become more affordable their popularity and utilization at the point-of-care (e.g., in the physician's office) has increased. For example, healthcare providers are currently using ultrasound machines in departments throughout the healthcare system for the purposes of procedural, diagnostic, and physical exams. The use of these imaging devices at the point-of-care and outside of the conventional radiology workflow has brought about the need for proper storage, documentation, billing, and peer review of these image studies.

Currently, the problem experienced by healthcare organizations with in-office imaging is that the organizations do not have an efficient and reliable method to capture and attach the correct patient metadata to image studies performed in the office. Many areas outside of radiology and cardiology acquire images at the point-of-care during an office visit and would benefit greatly from getting a DICOM order to their imaging device without purchasing and setting up a separate Radiology Information System (“RIS”) to manage the ordering, scheduling, patient reporting, and image management processes. Additionally, with in-office imaging, the resulting clinical images are often not properly stored in the electronic health record (“EHR”), leading to orphaned studies that remain in quarantine due to bad patient data or patient images stored on jump drives, CDs, and disks in an unsecure environment.

Thus, there remains a need to provide a medical image management system and workflow solution for medical imaging that occurs outside of the conventional radiology workflow.

As modalities started to support the digital display of images on workstations connected to the acquisition device, Picture Archiving and Communications Systems (PACS) came to market. These centrally store images and provide radiologists with the tools to read studies on networked computer monitors, replacing both film and modality workstations.

### SUMMARY OF THE DISCLOSURE

The present disclosure addresses the aforementioned drawbacks by providing a medical imaging workflow management system that includes a client, a database, and a worklist broker. The client is implemented with a hardware processor and a memory, and is configured to generate a medical imaging order comprising study metadata associated with an imaging device and a patient to be imaged by the imaging device. The database is in communication with the client to receive the medical imaging order from the client and store the medical imaging order therein. The worklist broker is implemented with a hardware processor and a memory and in communication with the database. The worklist broker is configured to generate a DICOM order by transposing the study metadata contained in the medical imaging order into a DICOM format; store the DICOM order to a worklist; receive a query associated with the medical imaging order from the imaging device; identify the medical imaging order in the worklist; retrieve the medical imaging order from the worklist; and send the DICOM order and metadata contained therein to the imaging device to be attached to images obtained therewith.

It is another aspect of the present disclosure to provide a medical imaging workflow management system that includes a worklist broker implemented with a hardware processor and a memory. The worklist broker is configured to receive a query from a medical imaging device and identify orders in response thereto; retrieve a medical imaging order from a database; transpose metadata contained in the medical imaging order to a DICOM format; and provide the transposed metadata to the imaging device to be attached to images acquired therewith.

It is another aspect of the present disclosure to provide a medical imaging workflow management system that includes a client implemented with a hardware processor and a memory. The client is configured to display image orders associated with medical images stored in a long term archive; identify medical images stored on the long term archive as being quarantined medical images; update metadata associated with the quarantined medical images; remove the quarantined medical images from quarantine; and reprocess a C-store of an image study to the long term archive when the metadata associated with the quarantined medical images has been updated.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1A is a block diagram of an example medical imaging workflow management system.

FIG. 1B is a block diagram of an example network that can be implemented in the medical imaging workflow management system of FIG. 1A.

FIGS. 2A and 2B show an illustration of an example database configured to store medical imaging orders and associated metadata.

FIG. 3 is a flowchart setting forth the steps of an example method for managing a medical imaging workflow using an orders first method.

FIG. 4 is a flowchart setting forth the steps of an example method for managing a medical imaging workflow using an images first method.

FIG. 5 is a flowchart setting forth the steps of an example method for processing quarantined images to update or otherwise correct metadata attached to those images.

FIG. 6 is a flowchart setting forth the steps of an example method for managing a medical imaging workflow using an orders first method in which non-DICOM images are retrieved from a data storage and are DICOM wrapped.

FIG. 7 is a block diagram of an example computer system that can implement systems, methods, and algorithms described in the present disclosure.

### DETAILED DESCRIPTION

Described here are systems and methods for managing medical images acquired in a point-of-care setting outside of a conventional radiology workflow. As will be described below, the medical imaging workflow management system of the present disclosure implements a dual workflow configuration, in which an “orders first” workflow and an “images first” workflow are implemented.

The dual workflows provided by the system described here allow clinicians to make their decision to image a patient either in advance via the “orders first” workflow, or on the fly via the “images first” workflow, thereby freeing the clinician to care for patients without worrying about whether an imaging order has been properly placed. The medical imaging workflow management system of the present disclosure provides a method for the clinician to later add order information to the images in an efficient manner. Alternatively, if there is no reason to keep the images that were obtained without an order first, the image study C-store job may be canceled by the user at the client.

In the “orders first” workflow, the medical imaging workflow management system of the present disclosure attaches the image order to a provider's pre-existing event and does not require the use of a Radiology Information System (“RIS”), thereby eliminating the need for making an additional device appointment and processing the exam through the RIS.

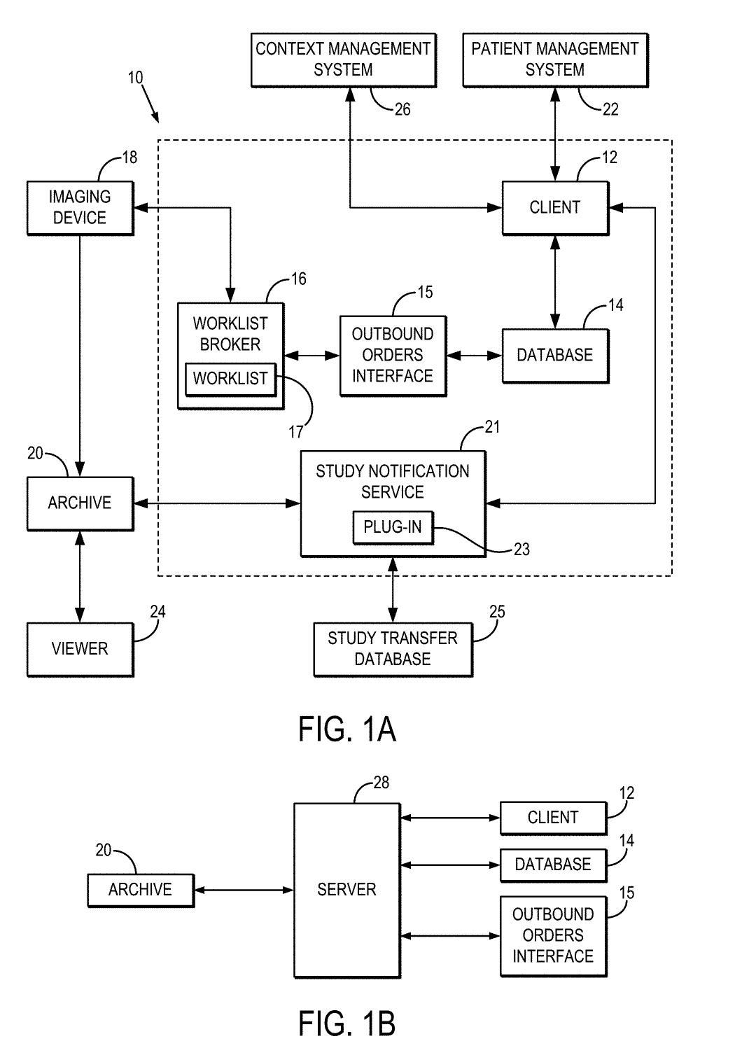

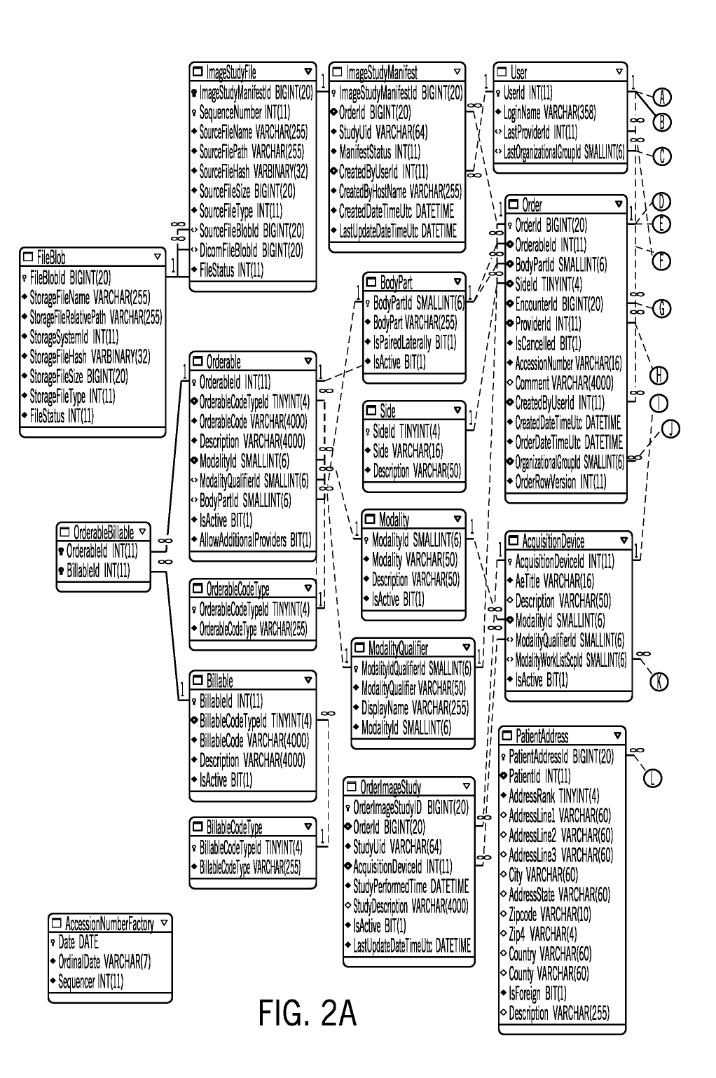

FIG. 1A illustrates an example medical imaging workflow management system 10. The system 10 includes a client 12 that communicates with a database 14, an outbound orders interface 15, and a worklist broker 16 to provide ordering, such as non-appointment ordering, of medical imaging studies with an imaging device 18 located in a clinical office or other point-of-care setting. As shown in FIG. 1B, communication between the client 12, the database 14, and the outbound orders interface 15 can be implemented via a server that is configured to operate as a service layer or middleware. The outbound orders interface 15 may implement, for example, HL7 standards. The imaging device 18 can include an ultrasound imaging device; an x-ray imaging device, including a digital radiography device, a mammography device, a computer tomography (“CT”) scanner, an x-ray fluoroscopy device, and so on; a magnetic resonance imaging (“MRI”) scanner; a nuclear medicine imaging device; an optical imaging device, including a camera; and so on.

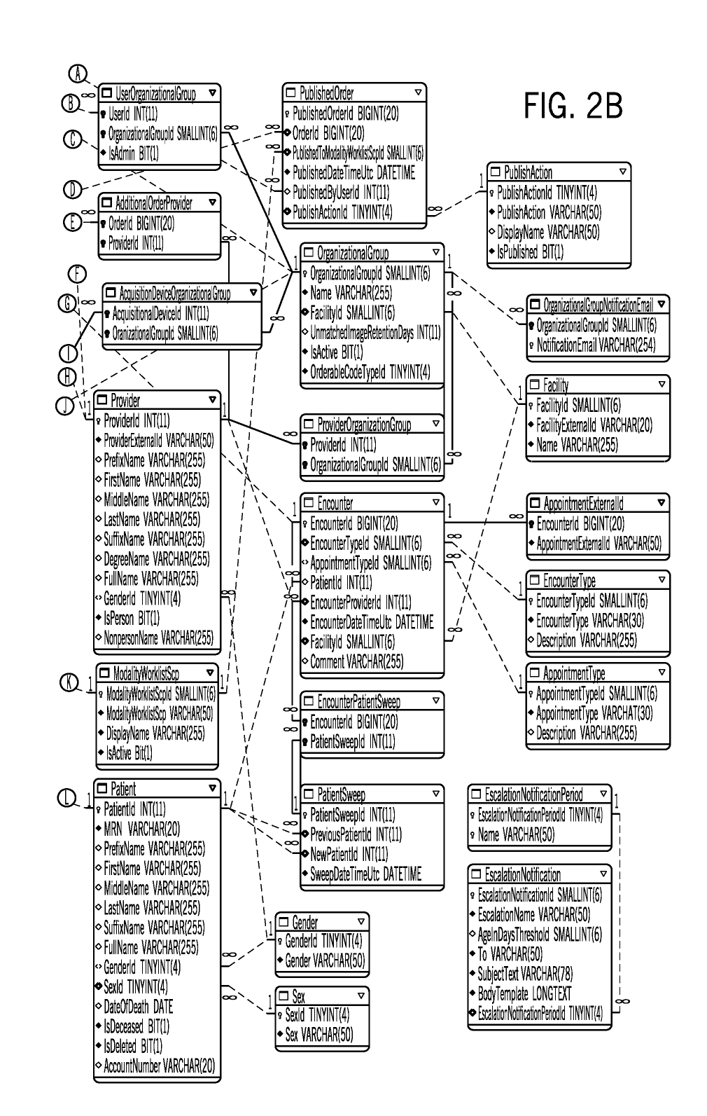

The client 12 can include a hardware processor, a memory, one or more inputs, and a display. In some examples, the client 12 can include a desktop computer, a laptop computer, a tablet device, a mobile device. The database 14 can be any suitable database for storing information such as medical imaging orders and associated study metadata. In some examples, the database can implement a SQL database. An example database 14 structure is illustrated in FIGS. 2A and 2B, which show the various metadata information that can be stored in an order generated by the client 12. The worklist broker 16 can generally include a hardware processor and a memory, and receives orders from the database 14 via an HL7 standard, which are then stored locally to the worklist 17 stored on the worklist broker 16.

The client 12 generally provides a user interface through which a user can communicate requests to the worklist broker 16, the imaging device 18, or both. For instance, a user can generate an order for medical imaging at the client 12 and this order is communicated to the database 14 for storage and sent to the worklist broker 16, from which it is later retrieved, when the imaging device 18 is used to image the patient associated with the order. To this end, the order includes study metadata input by the user at the client 12. Study metadata may include metadata information about the patient, the imaging procedure to be performed, provider information, the performing facility, and so on. The order can be a non-appointment order, such as may be generated in a point-of-care location where a clinician requests a medical imaging order at the time of evaluating a patient rather than in advance of the patient's appointment. This workflow provides an improvement over conventional ordering and scheduling workflows that require order placement through a RIS.

As shown in the example of FIG. 1, the imaging device 18 queries the worklist broker 16 to request an image study order. The worklist broker 16 identifies an order in the worklist 17 and retrieves the order that has been transposed into a Digital Imaging and Communications in Medicine (“DICOM”) format. For instance, when a new order is created at the client 12 the order is stored on the database 14 and sent (e.g., via HL7) to the worklist broker 16. The order sent to the worklist broker 16 from the database 14 can be a copy of the order, such that a copy of the order remains on the database 14. At the worklist broker 16, the order is transposed into a DICOM format and stored to the worklist 17. The worklist 17 may be a database, or other storage media, on the worklist broker 16 that keeps orders in DICOM format and which can be queried by an imaging device 18. The DICOM formatted order is then retrieved by the imaging device 18 where it is associated with the images acquired by the imaging device 18.

The images and attached order are then sent to an archive 20 that is in communication with the imaging device. The archive 20 can be a Picture Archiving and Communications System (“PACS”), a vendor neutral archive (“VNA”), a long term archive (“LTA”), or other suitable archive for storing medical images and associated metadata on a short-term or long-term basis. As shown in FIG. 1B, similar to the client 12, database 14, and outbound orders interface 15, communication with the archive 20 can be implemented via a server 28 that is configured to operate as a service layer or middleware.

In some instances, a user will acquire medical images with the imaging device 18 without having previously generated an order. In these instances, the imaging device 18 will query the worklist broker 16 and the worklist broker 16 will not find an associated order in the worklist 17 stored on the database 14. The images acquired with the imaging device 18 are thus sent to the archive 20 without study metadata being attached thereto.

The archive 20 implements an algorithm to verify the images received from the imaging device 18. The verification algorithm operates on device data contained in the DICOM header of the images received from the imaging device 18 to identify where an image study came from and to capture studies that may have been sent through without an order. At the start of the verification process, the archive 20 first alerts a study notification service 21 that a new study has arrived. In some embodiments, the study notification service 21 contains a plug-in 23 that queries the database 14 to locate a matching order with an accession number identical to the one found on the DICOM header. When a matching order is found, verification succeeds (e.g., the images are identified as containing study metadata from an imaging order), and the images are stored in the archive 20 using the study metadata data retrieved from the order by the worklist broker 16. A record of the completed study transfer is then stored in a study transfer database 25 that is in communication with the archive 20, and the order status on the client 12 is updated from “In Process” to “Complete”.

When a matching order is not found by the study notification service plug-in 23, the archive 20 accepts the images, but they are flagged and stored in a quarantined state. A record of the unverified study transfer is stored in the study transfer database 25 that is in communication with the archive 20.

The client 12 implements the order application, which queries the study transfer database 25 upon launch to find any unverified studies that originated from the set of devices configured for the current user's care team. The resulting “unverified” study list displays at the client 12 so that a user may attach an order after the image file is received.

The client 12 provides a user interface through which a user can view and analyze image study data stored on the archive 20. The user interface contains tools to create an order with patient metadata retrieved from the patient management system 22, which contains the healthcare institution's patient management records; match the order with a previously unverified study; reprocess the study transfer and verification steps at the archive 20; and successfully store the study. Alternatively, the user may cancel the study transfer and process a delete of an unverified study from the archive 20 from within the client 12. In some instances, if a user does not update or add the required study metadata to a quarantined image within a specified timeframe, the client 12 (e.g., the application at the client 12) will implement an algorithm to automatically notify member of the care team via an auto-generated message, such as an auto-generated email message.

The user interface can also contain tools to create an order with patient context data retrieved from a context management system 26 or application, which can be queried to retrieve patient context information from other clinical applications.

A viewer 24 can be in communication with the archive 20 to receive images from the archive 20 for viewing. The viewer 24 can provide an interface between the electronic medical record (“EMR”) and the archive 20.

The medical imaging workflow management system 10 described here can implement a thick client application on the client 12 and that works together with the database 14, the outbound orders interface 15, the worklist broker 16, and the archive 20 to create, manage, and store in-office imaging orders and related information. As such, the described system 10 can securely store and make accessible medical image studies in the patient's electronic medical record from DICOM-enabled imaging devices 18 located within the provider's office. In addition, the described system can display information and notifications from a connected archive 20, such as a PACS, VNA, or LTA, for unverified image studies that are stored in quarantine on that system.

Ancillary connections through an HL7 orders interface, an API/interface into the patient management system 22, the worklist broker 16, and the archive 20 study notification service 21 can facilitate the exchange of orders and study metadata needed between the described system 10, the archive 20, and the EHR.

Users can launch an application at the client 12 (e.g., the client application) to both place a new order and view any quarantined image studies from their area. Display of quarantined studies can be determined by a configurable component called “care team” in relation to the AE title of the client device the study was captured on. The connection of the system 10 to the patient management components of the EHR provides the ability to query for and select a patient, and search for and link to a pre-existing event. Selections for modality, exam description, organ, side, date and time of service, provider and facility as well as a free text comments box can combine with patient and event data to create an imaging order. The user can then publish this order to the database 14, which sends it to the worklist broker 16 for selection on an imaging device 18, or submit the order to the archive 20 for re-processing of a quarantined study with the updated metadata.

Additional views provided on the user interface of the client 12 can include a historical search for viewing and an ability to edit or cancel past image order entries stored on the database 14 that are not in a completed state.

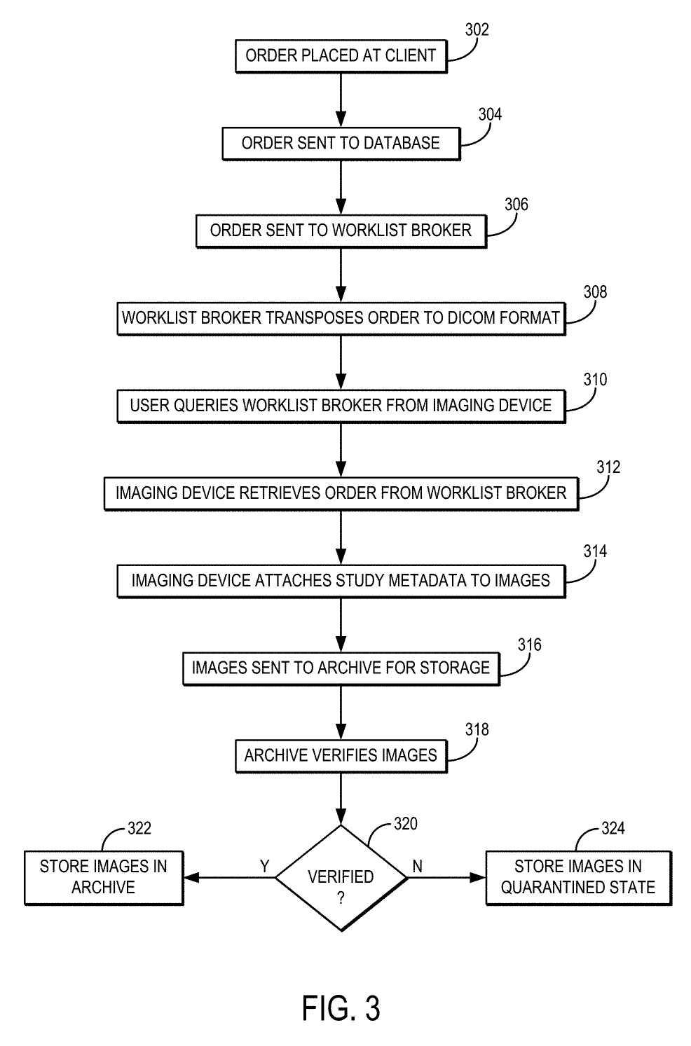

Referring now to FIG. 3, a flowchart is illustrated as setting forth the steps of an example method for managing the workflow of a point-of-care imaging study requested without the necessity of a RIS using an “orders first” workflow in which an imaging study order is generated prior to imaging the patient. As described above, the method includes a user placing an order at a client device, as indicated at step 302. As described above, the order may be a non-appointment order, or an order generated in preparation for a planned patient appointment. The order can be generated by the user at the client device as described above. As an example, the order is generated by the user entering data into a user interface. As another example, the client application can query a patient management system to retrieve metadata associated with a patient who has an existing appointment for an imaging study to generate an order. When generating or otherwise placing an order, a context management system or application can be queried to retrieve patient context information from other clinical applications to be attached to the order as metadata. As still another example, the order can be generated or otherwise placed in a third party system or EHR and then translated into a new order record at the server. For instance, the server can operate as a service layer between the client device or other systems, and the database. The server can generate a new order record from the third party order and store it to the database.

When completed, the order is sent from the client device to a database, as indicated at step 304. As described above, the database can be a SQL database or any other suitable data base for storing study metadata, such as other relational databases. The order is then sent to the worklist broker, as indicated at step 306 and is transposed to a DICOM format, as indicated at step 308. When the patient is ready to be imaged, the imaging device with which the patient will be imaged queries a worklist broker that has received and stored an order from the database, as indicated at step 310. In some instances, the imaging device can include a user interface that provides a display showing orders queued in the worklist, such that the user can select the appropriate order for the patient being imaged. In response to the query for an order, the worklist broker retrieves the appropriate order from the worklist, as indicated at step 312, which has transposed the metadata contained therein into a DICOM format. For instance, the client application sends the order from the database to the worklist broker via an HL7 protocol, which then transposes that data into a DICOM format and stores the order to its worklist. When queried by a connected image device, the worklist broker provides the DICOM-formatted metadata to the imaging device.

Images of the patient are acquired after the order has been retrieved, and the DICOM-formatted metadata received from the worklist broker is attached to the images, as indicated at step 314. The acquired images (and the attached metadata) are then communicated to an archive for storage, as indicated at step 316. The archive may include a PACS, VNA, LTA, or other suitable archive for storing medical images and associated data on a short-term or long-term basis.

When the images and attached metadata are received by the archive, the archive implements a verification algorithm to verify the status and metadata content of the images. If the images are verified, as determined at decision block 320, they are stored in the archive, as indicated at step 322. If the images are not verified (e.g., if there is metadata missing from the images or incorrect metadata attached to the images) then the archive stores the images, but flags the images as being in a quarantined state, as indicated at step 324. As will be described below, the images stored in the quarantined state are reported to the client device to update or correct the metadata attached to or missing from the quarantined images.

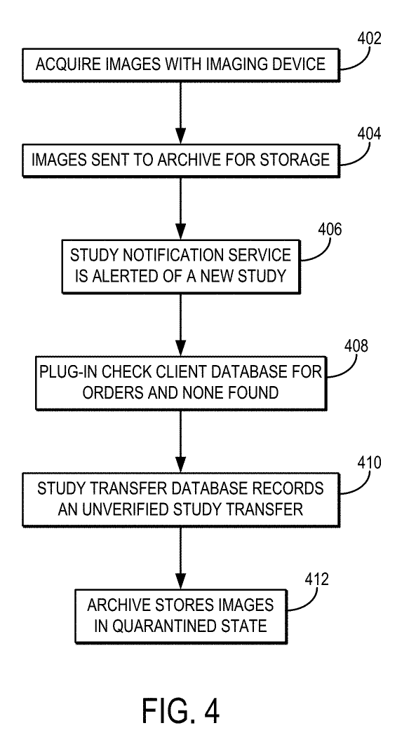

Referring now to FIG. 4, a flowchart is illustrated as setting forth the steps of an example method for managing the workflow of an in-office, or point-of-care, imaging study requested without the necessity of a RIS using an “images first” workflow in which an imaging order has not been generated for the imaging study. This method is useful for situations where a clinician wants to image a patient on the fly, whether as part of routine evaluation or in an emergent situation. Images are acquired with the imaging device, or are otherwise retrieved from a memory or other data storage, as indicated at step 402. The acquired images are then communicated to an archive for storage, as indicated at step 404. The archive may include a PACS, VNA, LTA, or other suitable archive for storing medical images and associated data on a short-term or long-term basis. 100421 These images are acquired without an imaging order and the associated metadata. Under normal circumstances, these images would likely be orphaned in the healthcare institution's archive; however, using the methods described here such images can be efficiently and reliably processed. The study notification service is alerted of a new study, as indicated at step 406. A plug-in implemented by the study notification service checks the client database for orders associated with the new study and verifies that no orders are found, as indicated at step 408. The study transfer database then records the new study as an unverified study transfer, as indicated at step 410, and the images associated with this unverified study are stored in a quarantined state, as indicated at step 412. As will be described below, images stored in the quarantined state are reported to the client device to update or correct the metadata attached to or missing from the quarantined images.

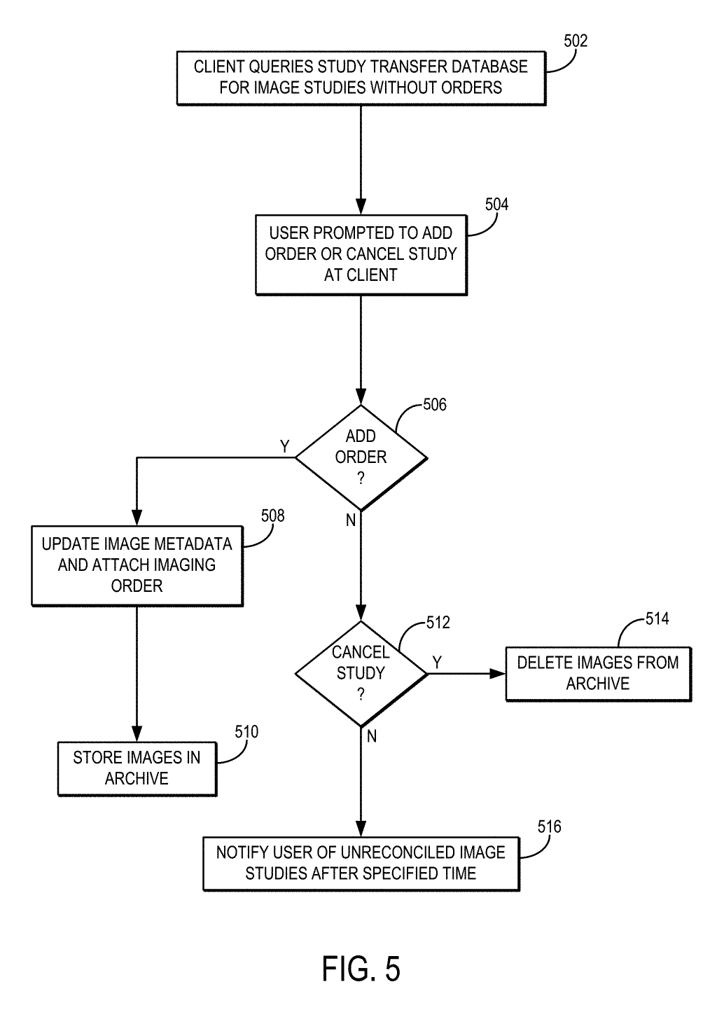

Referring now to FIG. 5, a flowchart is illustrated as setting forth the steps of an example method for processing images in a quarantined state. Upon launch, the client queries the ancillary study transfer database of the archive on which the quarantined images are stored. The resulting list of unverified studies is displayed on the client device for the quarantined images to have their metadata updated or otherwise corrected by a user, as indicated at step 502. Based on the client's query, the user is prompted to add order information to a study in the unverified studies list, or to cancel the study, as indicated at step 504. If the user selects to add an order to a study in the unverified study list, as determined at decision block 506, then the user is provided with an interface to search for patient metadata and to create an order to be attached to the unverified study. As one example, the user can operate the client device to query the healthcare institution's patient management system for metadata that can be attached to the quarantined images associated with the unverified study. As another example, context management systems or applications can be used to retrieve context patient information from other clinical applications, and to use the context patient information as metadata that can be attached to the quarantines images associated with the unverified study. The metadata for the quarantined images are then updated the imaging order attached to the unverified study, as indicated at step 508. The images are then removed from quarantine and are moved to the archive for successful storage, as indicated at step 510.

When the user selects to cancel the unverified study, as determined at decision block 512, the images are deleted from the archive, as indicated at step 514. If the user decides to do nothing, the data in the unverified study is not updated. As a result, the user is notified automatically that they must reconcile or cancel, as indicated at step 516. This notification may be presented to the user immediately, or after a specified amount of time.

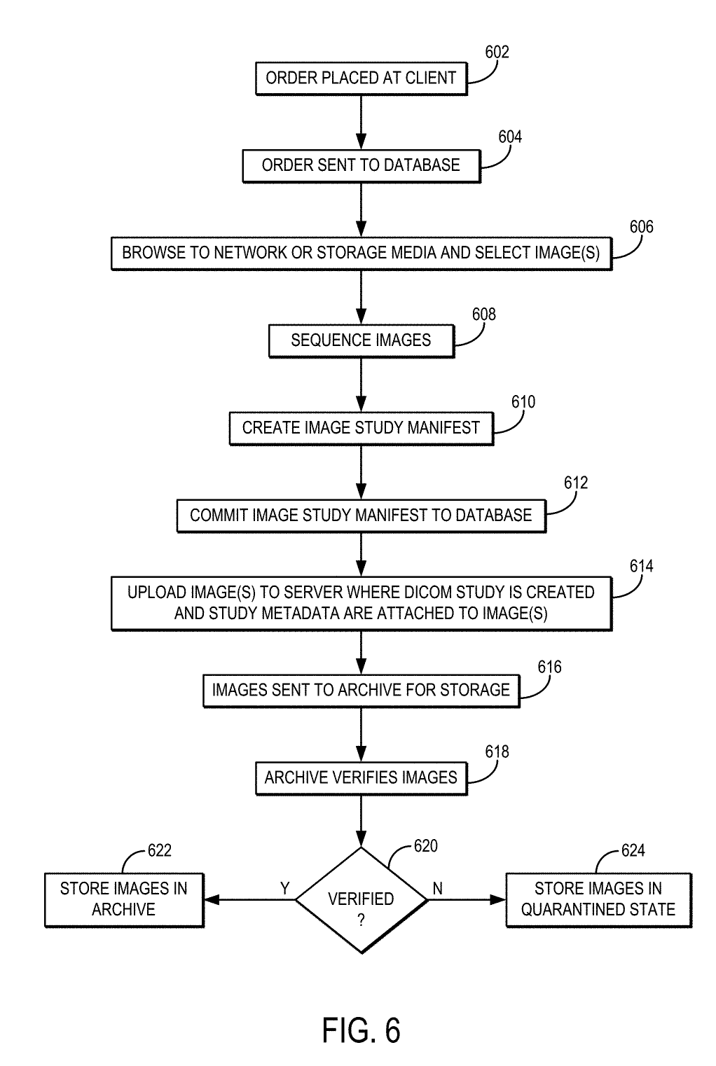

Referring now to FIG. 6, a flowchart is illustrated as setting forth the steps of an example method for managing the workflow of a point-of-care imaging study requested without the necessity of a RIS in which the image(s) acquired during the image study are stored in a non-D IC OM format. As described above, the method includes a user placing an order at a client device, as indicated at step 602. As described above, the order may be a non-appointment order, or an order generated in preparation for a planned patient appointment. The order can be generated by the user at the client device as described above. As an example, the order is generated by the user entering data into a user interface. As another example, the client application can query a patient management system to retrieve metadata associated with a patient who has an existing appointment for an imaging study to generate an order. When generating or otherwise placing an order, a context management system or application can be queried to retrieve patient context information from other clinical applications to be attached to the order as metadata. As still another example, the order can be generated or otherwise placed in a third party system or EHR and then translated into a new order record at the server. For instance, the server can operate as a service layer between the client device or other systems, and the database. The server can generate a new order record from the third party order and store it to the database.

When completed, the order is sent from the client device to a database, as indicated at step 604. As described above, the database can be a SQL database or any other suitable data base for storing study metadata, such as other relational databases. The order can then be processed as described above.

Images of the patient are acquired before the order has been placed and are stored in a non-DICOM format in a network file location or other storage media. The non-DICOM images can be image formatted as JPG, BMP, PDF, or other digital image file types. When entering an order, a user interface is presented to the user to browse to a computer file or network file location in order to select an image, as indicated at step 606.

The selected images are then ordered into the desired sequence, as indicated at step 608. An image study manifest is then created, as indicated at step 610, and then committed to the database, as indicated at step 612. For instance, the image study manifest can be committed to the database by building the data set associated with the image study manifest and storing that data to a table in the database. An example of an image study manifest data structure is illustrated as the “ImageStudyManifest” data entry in FIG. 2A; although, it will be appreciated that other image study manifest data structures can also be implemented. The images and the attached metadata are then processed to attach study metadata and form a DICOM image study, as indicated at step 614. The acquired images (and the attached metadata) are then uploaded to the server by the client and then communicated to an archive for storage, as indicated at step 616. The archive may include a PACS, VNA, LTA, or other suitable archive for storing medical images and associated data on a short-term or long-term basis.

When the images and attached metadata are received by the archive, the archive implements a verification algorithm to verify the status and metadata content of the images. If the images are verified, as determined at decision block 620, they are stored in the archive, as indicated at step 622. If the images are not verified (e.g., if there is metadata missing from the images or incorrect metadata attached to the images) then the archive stores the images, but flags the images as being in a quarantined state, as indicated at step 624. As described above, the images stored in the quarantined state are reported to the client device to update or correct the metadata attached to or missing from the quarantined images.

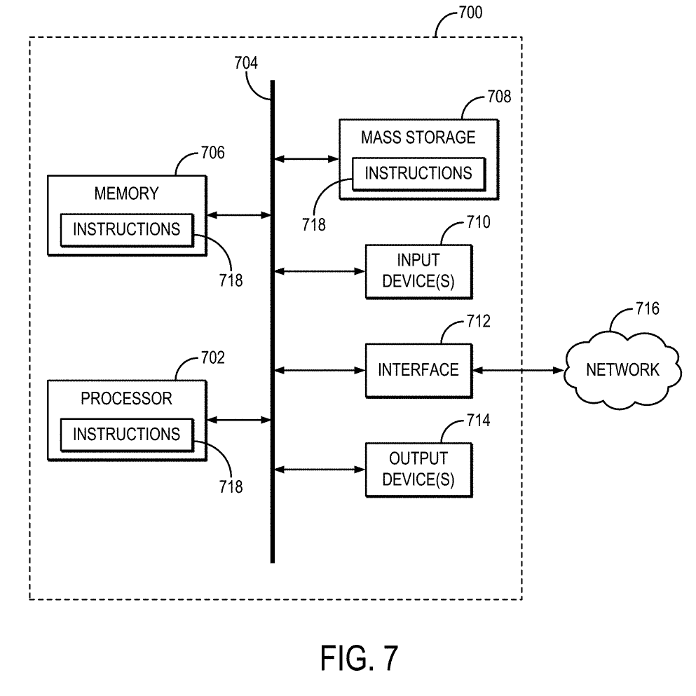

FIG. 7 is a block diagram illustrating an example of a computer system 700 that can implement systems, methods, and algorithms described here. The computer system 700 can include a processor 702 that is coupled to an interconnect 704, which may be an interconnection bus or the like. As an example, the processor 702 can be any suitable processor, processing unit, or microprocessor. Furthermore, the processor 702 may include a single processor or multiple different processors that are coupled to the interconnect 704.

The processor 702 is coupled to a memory 706 via the interconnect 704. The memory 706 can include any type of volatile memory, non-volatile memory, or combinations of both, including static random access memory (“SRAM”), dynamic random access memory (“DRAM”), flash memory, read-only memory (“ROM”), and so on.

The computer system 700 also includes a mass storage device 708, one or more input devices 710, an interface 712, and one or more output devices 714 that are connected to the interconnect 704. The one or more input devices 710 may include a keyboard, a mouse, a touch screen display, and so on. The interface 712 may be any suitable interface for wired or wireless communication between the computer system 700 and another computer system via a network 716. The one or more output devices 714 may include a display or the like.

The mass storage device 708 can include a machine-readable medium on which is stored one or more sets of data structures and instructions 718 (e.g., software) embodying or utilized by any one or more of the systems, methods, or algorithms described here. The instructions 718 may also reside, completely or at least partially, within the memory 706 or a local memory within the processor 702. The instructions 718 may also be transmitted or received over the network 716 and received by the computer system 700 via the interface 712.

The present disclosure has described one or more preferred embodiments, and it should be appreciated that many equivalents, alternatives, variations, and modifications, aside from those expressly stated, are possible and within the scope of the invention.

1. A medical imaging workflow management system, comprising:
a client implemented with a hardware processor and a memory, the client being configured to generate a medical imaging order comprising study metadata associated with an imaging device and a patient to be imaged by the imaging device;
a database in communication with the client to receive the medical imaging order from the client and store the medical imaging order therein;
a worklist broker implemented with a hardware processor and a memory and in communication with the database, the worklist broker being configured to:
generate a DICOM order by transposing the study metadata contained in the medical imaging order into a DICOM format;
store the DICOM order to a worklist;
receive a query associated with the medical imaging order from the imaging device;
identify the medical imaging order in the worklist;
retrieve the medical imaging order from the worklist; and
send the DICOM order and metadata contained therein to the imaging device to be attached to images obtained therewith.

2. The medical imaging workflow management system as recited in claim 1, further comprising an interface operably connected to an archive containing an ancillary study transfer database and containing images obtained with the imaging device, and further wherein the client is configured to process a query to the ancillary study transfer database of the archive and return a list of unverified studies when the DICOM order metadata attached to the images is one of missing, incorrect, or incomplete, and displays the list of unverified studies to a user.

3. The medical imaging workflow management system as recited in claim 2, wherein the client is configured to:
query for unverified studies from the archive;
request updated metadata from a connected patient management system;
update a DICOM header in the images with the updated metadata; and
send the images to the archive with the updated metadata attached so the images can be properly verified and permanently stored.

4. The medical imaging workflow management system as recited in claim 3, wherein the client is configured to request the updated metadata by providing a user interface to a user via which the user inputs an order to a previously unverified image study and applies patient metadata obtained from the connected patient management system.

5. The medical imaging workflow management system as recited in claim 2, wherein the notification is sent to the client in response to a verification algorithm implemented by the archive to verify the DICOM-formatted metadata attached to the images.

6. The medical imaging workflow management system as recited in claim 5, wherein the verification algorithm flags the images as being in a quarantined stated when the DICOM-formatted metadata are identified as one of missing, incorrect, or incomplete.

7. The medical imaging workflow management system as recited in claim 2, wherein the archive is at least one of a picture archiving and communications system (PACS), a vendor neutral archive (VNA), or a long term archive (LTA).

8. The medical imaging workflow management system as recited in claim 1, wherein the hardware processor and the memory that implement the client form a part of the imaging device.

9. The medical imaging workflow management system as recited in claim 1, wherein the client is in communication with a patient management system and is configured to generate the medical imaging order by sending a query to the patient management system and receives in response thereto metadata associated with the patient and an existing appointment associated for the patient.

10. The medical imaging workflow management system as recited in claim 1, further comprising an outbound orders interface implemented with a hardware processor and a memory, wherein the outbound orders interface sends the order from the database to the worklist broker using an HL7 standard.

11. The medical imaging workflow management system as recited in claim 1, wherein the client is in communication with a context management system and is configured to generate the medical imaging order by sending a query to the context management system and receives in response thereto metadata associated with the patient.

12. The medical imaging workflow management system as recited in claim 1, wherein the client receives a third party order from a different computer system and creates the medical imaging order from the third party order.

13. A medical imaging workflow management system, comprising:
a client implemented with a hardware processor and a memory, the client being configured to:
display image orders associated with medical images stored in a long term archive;
identify medical images stored on the long term archive as being quarantined medical images;
update metadata associated with the quarantined medical images;
remove the quarantined medical images from quarantine; and
reprocess a C-store of an image study to the long term archive when the metadata associated with the quarantined medical images has been updated.

14. The medical imaging workflow management system as recited in claim 13, wherein the client is configured to update the metadata associated with the quarantined medical images by providing a user interface via which a user associates updated metadata from a connected patient management system.

15. The medical imaging workflow management system as recited in claim 13, wherein the client is configured to update the metadata associated with the quarantined medical images by:
querying a patient management system in communication with the client;
receiving additional metadata from the patient management system in response to being queried by the client; and
update the metadata associated with the quarantined medical images with the additional metadata.

16. A medical imaging workflow management system, comprising:
a worklist broker implemented with a hardware processor and a memory, the worklist broker being configured to:
receive a query from a medical imaging device and identify orders in response thereto;
retrieve a medical imaging order sent from a database;
transpose metadata contained in the medical imaging order to a DICOM format; and
provide the transposed metadata to the imaging device to be attached to images acquired therewith.

17. A medical imaging workflow management system, comprising:
a client implemented with a hardware processor and a memory, the client being configured to generate a medical imaging order comprising study metadata associated with an imaging device and a patient previously imaged by the imaging device;
a database in communication with the client to receive the medical imaging order from the client and store the medical imaging order therein;
wherein the client is configured to:
retrieve non-DICOM images from a data storage location in response to a user input when generating the medical imaging order, wherein the non-DICOM images have been previously acquired by the imaging device from the patient;
create an image study manifest based on the non-DICOM images;
commit the image study manifest to the database; and
generate a DICOM image study based on the image study manifest by attaching the study metadata to the non-DICOM images.

18. The medical imaging workflow management system as recited in claim 17, further comprising an interface operably connected to an archive, and wherein the client is configured to store the DICOM image study in the archive.
